(19) United States
(12) Patent Application Publication
(10) Pub. No.: US 2007/0183986 A1
(43) Pub. Date: Aug. 9, 2007

(54) Methods and systems for mixing a multi-part fluoride varnish composition

(75) Inventors: Peter M. Allred, Riverton, UT (US); Jeff Wagner, Salt Lake City, UT (US); Dan E. Loveridge, Sandy, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Ultradent Products, Inc., South Jordan, UT (US)

(21) Appl. No.: 11/348,055

(22) Filed: Feb. 6, 2006

(51) Int. Cl. A61K 8/21; A61K 8/30

(52) U.S. Cl. 424/52

(57) ABSTRACT

Methods and related systems for mixing a multi-phase fluoride varnish composition preparatory to applying it to a person's teeth. The method includes the steps of providing a multi-phase fluoride varnish composition including a liquid hydrophobic adhesive phase and a solid fluoride salt (e.g., sodium fluoride) phase that is substantially insoluble in the liquid adhesive phase, and applying turbulence to the multi-phase composition within a closed vessel in order to substantially suspend the solid fluoride salt phase within the liquid hydrophobic adhesive phase while preventing spillage of the composition.

### BACKGROUND OF THE INVENTION

1. The Field of the Invention

The present invention generally relates to the field of dentistry, and more particularly to methods and systems for mixing a multi-phase fluoride varnish composition.

2. The Relevant Technology

In the field of dentistry, a fluoride composition is often applied to a patient's teeth as part of a routine dental cleaning. Fluoride compositions are often applied to children's teeth, although they are sometimes applied to an adult's teeth also. Application of fluoride has been widely recognized as a method of preventing tooth decay.

According to one method, a fluoride gel composition is dispensed within a dental tray, after which the tray is placed over the patient's teeth. The tray holds the fluoride gel composition adjacent to the patient's teeth, and after a desired amount of time (e.g., a few minutes or less), the tray is removed and the remaining gel composition is rinsed off the patient's teeth. Because of the short exposure time of the patient's teeth to the fluoride gel composition (e.g., typically a couple minutes or less), alternative methods that allow increased exposure time (and thus more effective tooth decay prevention) have been developed.

One such method involves application of a fluoride varnish composition to a patient's teeth. A typical fluoride varnish composition includes a fluoride salt dispersed within a sticky, adhesive hydrophobic varnish material. The fluoride varnish composition is typically stored within a foil covered tray, allowing the dental practitioner to peel away the cover, dip a brush into the tray, and brush the mixture onto the patient's teeth. Once applied, the varnish composition adheres to the teeth, allowing a longer exposure time (e.g., as long as, or greater than 2 hours) before the varnish composition is worn away by the action of saliva and/or the patient's tongue.

Although such fluoride varnish compositions allow for longer exposure times, existing methods of fluoride varnish application have disadvantages. Varnish components are typically multi-phase such that the fluoride salt is insoluble, causing the solid fluoride salt phase to settle out of the mixture during storage. Because of this, the dental practitioner is required to stir the mixture (e.g., with a stirring stick) prior to application. This mixing process requires use of an extra tool (i.e., a stirring stick), is inefficient and wasteful (i.e., the varnish adhering to the stick is unavailable for placement onto a person's teeth), and fails to ensure complete mixing. In addition, stirring typically results in the composition slopping over the edges of the small tray, thereby causing further waste and mess. Shaking the foil covered tray prior to opening is ineffective at suspending the solid fluoride salt phase because of the very high viscosity of the composition (e.g., typically at least about 1000 centipoise).

It would be an improvement in the art to provide a method and associated mixing system that would quickly and easily allow a dental practitioner to mix a multi-phase fluoride varnish composition prior to application without the mess, inefficiency, and waste associated with existing methods.

### SUMMARY OF THE INVENTION

The present invention is directed to a method and related system for mixing a multi-phase fluoride varnish composition preparatory to applying it to a person's teeth. The method includes the acts of providing a multi-phase fluoride varnish composition including a liquid hydrophobic adhesive phase and a solid fluoride salt (e.g., sodium fluoride) phase that is substantially insoluble in the hydrophobic adhesive phase, and applying turbulence to the composition within a closed vessel in order to substantially suspend the solid fluoride salt phase within the hydrophobic adhesive phase while preventing spillage and waste of the varnish composition. The manner of applying turbulence should be capable of suspending the solid fluoride salt phase within a high viscosity liquid hydrophobic adhesive phase (e.g., the multi-phase composition typically has a viscosity of at least about 1000 centipoise).

The inventive method and system advantageously allows the fluoride salt to be suspended within the varnish composition while eliminating any potential that the varnish composition may be spilled. Also advantageously, the method and system eliminates waste and easily ensures good mixing of the insoluble salt within the varnish composition without requiring the use of any additional tools (e.g., a stirring stick).

The hydrophobic adhesive phase of the fluoride varnish composition may comprise any non-toxic sticky material. Examples include, but are not limited to, shellac and/or colophonium. In addition to the hydrophobic adhesive, the composition may include one or more solvents (e.g., ethanol), thickeners, sweeteners, and/or flavorants.

In one example, the closed vessel in which the composition is mixed may comprise a hollow cylindrical container having an inner wall, and a plug slidably disposed within the hollow cylindrical container. The plug forms a seal against the inner wall, and is operable to provide turbulence to a quantity of the multi-phase fluoride varnish composition that is contained within the container. In one example, the hollow cylindrical container and plug may comprise a syringe barrel and a syringe plunger, respectively, of a first syringe.

One such apparatus may include a first syringe and a second syringe coupled to each other, while each syringe may include a barrel and a plunger slidably disposed within the barrel so as to allow a user to express the fluoride varnish composition from the barrel of the first syringe into the barrel of the second syringe. This configuration allows the dental practitioner to cycle the fluoride varnish composition back and forth as many times as desired under turbulent conditions so as to mix the multi-phase composition and substantially suspend the fluoride salt within the hydrophobic adhesive component. Suspension of the solid fluoride salt phase advantageously provides for fairly even distribution of the fluoride salt within the varnish composition as it is applied to a person's teeth.

Another closed vessel mixing system includes a first syringe coupled to a flexible bladder apparatus, allowing the multi-phase fluoride varnish composition to be cycled back and forth so as to apply turbulence and substantially suspend the fluoride salt. Another example of an alternative closed vessel mixing system includes a first syringe coupled to a spring loaded barrel, allowing the composition to be cycled back and forth so as to substantially suspend the fluoride salt. The multi-phase fluoride varnish composition may be cycled back and forth as many times as desired in order to produce substantial suspension of the fluoride salt within the liquid hydrophobic phase. For example, the fluoride salt may be substantially suspended by cycling the varnish composition back and forth between about two and about fifteen times, preferably between about three and about twelve times and more preferably between about five and about ten times. The inventive systems advantageously provide for suspension of the solid fluoride salt phase within the liquid adhesive phase while eliminating any potential that the varnish composition may be spilled.

Once the multi-phase fluoride varnish composition has been agitated so as to suspend the fluoride salt, the composition may be applied to a person's teeth. For example, when using a syringe-to-syringe mixing apparatus, the syringes may be decoupled and an applicator tip may be coupled to the delivery end of the syringe containing the fluoride varnish composition. Similarly, the flexible bladder or spring loaded barrel in alternative embodiments can be decoupled from the syringe and replaced with an applicator tip. The multi-phase fluoride varnish composition may then be applied to a person's teeth (e.g., by painting with a flocked applicator tip).

The fluoride varnish composition may be formulated so as to advantageously provide a desired aesthetic appearance on the surface of the person's teeth. For example, the varnish may be naturally colored so as to blend in with the person's teeth, or may include a colorant that provides a visual contrast against tooth tissue when applied and dried. Such a contrast advantageously allows a dental practitioner to more easily determine where the fluoride varnish has been applied. In addition, such a contrast may further allow a person to visually ascertain when the fluoride varnish has worn off.

The fluoride varnish composition may remain adhered to the person's teeth for a desired length of time. According to one embodiment, the fluoride varnish composition may advantageously remain adhered to the person's teeth so as to provide fluoride treatment for at least about eight hours, preferably for at least about one day and more preferably for at least about two days. Formulating the composition so as to remain adhered for an extended time (e.g., at least about eight hours) provides for an extended treatment time, which is a distinct advantage over compositions and methods that may provide fluoride treatment for a much shorter time period.

Furthermore, the closed vessel may advantageously be configured so as to contain a single use quantity of the multi-phase fluoride varnish composition. Such a system advantageously provides a single use system that can be provided to the dental practitioner as a unidose system which can be mixed and applied to the teeth of a single patient, after which the empty mixing apparatus can be discarded.

These and other benefits, advantages and features of the present invention will become more full apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of an exemplary two-syringe closed vessel mixing system that contains a multi-part fluoride varnish composition;

FIGS. 2A-2B depict the exemplary closed vessel mixing system of FIG. 1 being used to cycle the multi-part fluoride varnish composition back and forth;

FIGS. 3A-3B depict an alternative closed vessel mixing system including a syringe coupled to a flexible bladder;

FIGS. 4A-4C illustrate another alternative closed vessel mixing system including a syringe coupled to a spring loaded barrel;

FIGS. 5A-5B illustrate a syringe (e.g., from any of the mixing systems of FIGS. 1-4) being coupled to a flocked applicator tip; and

FIG. 6 illustrates the application of a multi-phase fluoride varnish composition to a tooth using a flocked applicator tip.

### DETAILED DESCRIPTION OF THE PREFERRED EMBODIMENTS

### I. INTRODUCTION

A detailed description of the invention will now be provided with specific reference to Figures illustrating various exemplary embodiments. It will be appreciated that like structures will be provided with like reference designations.

The present invention is directed to a method and associated system for mixing a multi-phase fluoride varnish composition preparatory to applying it to a person's teeth. The inventive method includes the steps of providing a multi-phase fluoride varnish composition including a liquid hydrophobic adhesive phase and a solid fluoride salt phase that is substantially insoluble in the liquid hydrophobic adhesive phase, and applying turbulence to the composition within a closed vessel in order to substantially suspend the solid fluoride salt within the liquid hydrophobic adhesive phase while preventing spillage of the composition. The inventive method and system advantageously allows the solid fluoride salt to be suspended within the varnish composition while eliminating any potential that the varnish composition may be spilled. Also advantageously, the method and system easily ensures good mixing of the insoluble salt within the varnish composition without requiring the use and associated mess of any additional tools (e.g., a stirring stick).

### II. THE MULTI-PHASE FLUORIDE VARNISH COMPOSITION

The multi-phase fluoride varnish composition includes a solid fluoride salt phase and a liquid hydrophobic adhesive phase. The liquid adhesive phase may comprise any non-toxic sticky material. Examples of such materials include shellac and/or colophonium. Commercially available shellac may be provided with a solvent (e.g., ethanol). One such shellac, known as Refined Pharmaceutical Glaze, is available from Mantrose-Haeuser Co., Inc., located in Westport, Conn. Colophonium (also known as rosin) refers to a class of sticky, non-synthetic naturally derived adhesive resins (e.g., typically derived from various species of pine). Colophonium typically includes a substantial fraction of resin acid components that are isomeric with abietic acid (C₂₀H₃₀O₂). Examples of colophonium also may include dihydroabietic acid (C₂₀H₃₂O₂) and/or dehydroabietic acid (C₂₀H₂₈O₂). Colophonium may range from black to colorless, is typically pale yellow to amber in color, has a density of about 1.07-about 1.09 g/cm³, and an acid number of not less than about 150. It is typically substantially insoluble in water, and freely soluble in alcohol, benzene, ether, glacial acetic acid, oils, and carbon disulfide. Various materials that are individually referred to as “colophonium” include Canadian balsam, Olibanum balsam, Elemi resin, Opopanax resin, Tolu balsam, Peruvian balsam, and Poly Pale resin, which is a partially dimerized rosin available from Eastman Chemical, located in Kingsport, Tenn. One or more adhesive components may be combined together to form the liquid hydrophobic adhesive phase. For example, it may be particularly advantageous to include both shellac and a colophonium in the liquid adhesive phase. It has been found that colophonium advantageously modifies the otherwise lower viscosity and runniness of the shellac, giving body to the overall composition and increasing its viscosity. Increased viscosity is advantageous during application of the composition, as it reduces or eliminates any tendency of the composition to “run” once applied to the teeth. One particularly preferred combination is Refined Pharmaceutical Glaze (about ⅓ shellac and ⅔ ethanol) and Poly Pale resin. The liquid adhesive phase advantageously allows the composition to adhere to a person's teeth so as to provide a fluoride treatment for as long as the composition remains adhered to the teeth. In addition, many liquid adhesive components naturally have a color to them (e.g., yellow). This natural coloring may be advantageous as it imparts a distinct and visible color contrast to the composition when viewed adjacent the teeth to which the composition is applied. Although advantageous for practical reasons, some patients (for aesthetic reasons) may prefer a composition that does not provide a visual contrast against the teeth to which the composition is applied. For this reason, the composition may be colorless (i.e., clear) or of a color (e.g., tooth-colored) so as to blend in with the person's teeth in appearance.

The fluoride varnish composition may further include one or more solvents (e.g., ethanol), one or more sweeteners, one or more flavorants, one or more colorants, and/or one or more thickeners. Ethanol is a particularly preferred solvent as it is non-toxic, although other solvents (e.g., acetone or isopropyl alcohol) could alternatively be used. If included, the solvent typically evaporates away after the composition is applied to the teeth. Examples of preferred flavorants include bubble gum, peach, tropical punch, grape, watermelon, cinnamon, methyl salicylate, natural wintergreen, cool mint, and/or creme de menthe. Examples of preferred sweeteners include sucralose and xylitol, although sodium saccharine and/or aspartame could also be used. Sucralose is made by chlorinating sucrose. Xylitol is a sugar alcohol. Sucralose and xylitol are particularly preferred as there is no evidence that either has any carcinogenic effect (as opposed to saccharine). In addition, sucralose and xylitol are particularly preferred over aspartame as the inventors have found that aspartame has less of a sweetening effect, and therefore more aspartame is required to achieve the same sweetness as a lower amount of sucralose or xylitol. Furthermore, xylitol has been found to exhibit antimicrobial effects.

Fumed silica is a preferred optional thickener, as it provides handling properties to the multi-phase composition that are particularly advantageous. The addition of fumed silica has been found to advantageously reduce the tendency of the composition to become stringy during handling. In other words, when attempting to stop the flow of the multi-phase composition (e.g., from a nozzle in a packaging operation or while dispensing from a syringe nozzle), the composition may have a tendency to form long spider-web like strings or threads (e.g., much like honey or baby slobber). Reducing this tendency provides handling properties that are not only advantageous during manufacture and packaging, but also during dispensing and use of the composition. One commercially available fumed silica product is AEROSIL 200, available from Degussa.

Advantageously, the fluoride varnish composition is sufficiently viscous so as to allow the practitioner to brush or otherwise apply the composition onto a patient's teeth without having to worry about the composition running or dripping off. Preferably the composition has a viscosity (under application temperature conditions) of at least about 1000 centipoise, more preferably between about 1300 and about 3500 centipoise, and most preferably between about 1700 and about 2800 centipoise. In addition, the composition is also preferably thixotropic (i.e., it becomes more fluid when under a disturbing shear force). Such a composition is more easily spread during application, and because of its high viscosity and thixotropic characteristics, any tendency of the composition to otherwise run or drip once applied is minimized and/or prevented. Although a viscosity of about 1000 centipoise provides an adequate degree of viscosity to minimize any tendency of the composition to run or drip, higher viscosities (e.g., at least about 1300 centipoise, more preferably at least about 1700 centipoise) provide much improved control to minimize and/or prevent the composition from running or dripping.

The solid fluoride salt phase may comprise any suitable fluoride salt, an example of which is sodium fluoride. In one example, the solid fluoride salt phase may be included in the composition in an amount between about 0.1% and about 10% by weight of the multi-phase fluoride varnish composition, preferably between about 1% and about 8% by weight of the multi-phase fluoride varnish composition, and more preferably between about 3% and about 7% by weight of the multi-phase fluoride varnish composition.

### III. EXEMPLARY MIXING SYSTEMS AND METHODS

In broad terms, mixing systems according to the invention include a closed vessel for holding a multi-phase fluoride varnish composition. A quantity of the multi-phase fluoride varnish composition, including a liquid hydrophobic adhesive phase and a solid fluoride salt phase that is insoluble in the liquid adhesive phase is contained within the closed vessel. The system further comprises means for providing turbulence to the multi-phase fluoride varnish composition within the closed vessel so as to substantially suspend the solid fluoride salt phase within the liquid adhesive phase. The means for providing turbulence does so while preventing spillage and waste of the composition, and is effective (e.g., as opposed to attempting suspension by shaking a high viscosity varnish composition within a foil covered tray prior to opening).

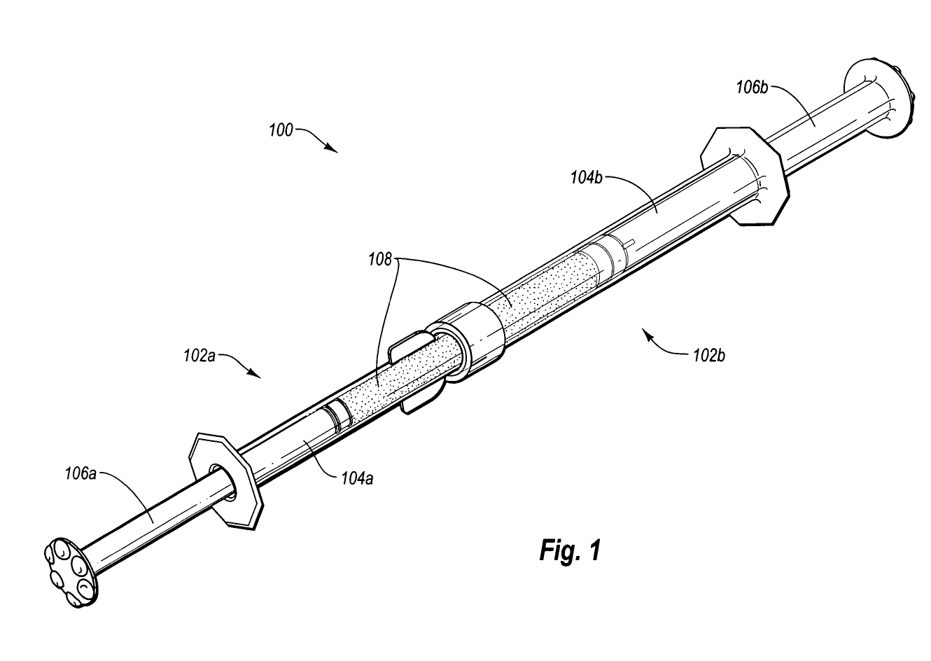

The system advantageously allows the solid fluoride salt phase to be suspended within the varnish composition, which provides for more even distribution of the fluoride onto the teeth during application. Suspension of the solid fluoride salt phase is accomplished while eliminating any potential that the composition may be spilled. In some exemplary systems, the closed vessel comprises a hollow cylindrical container including an inner wall. A plug is slidably disposed within the hollow cylindrical container, forming a seal against the inner wall. The plug is one example of means for providing turbulence for suspending the solid fluoride salt phase within the liquid adhesive phase. In one example, the hollow cylindrical container may be a syringe barrel of a first syringe while the plug may be a syringe plunger of a first syringe, such that the closed vessel comprises at least a first syringe. The mixing systems illustrated in FIGS. 1-4C are examples of such systems including a first syringe.

FIG. 1 illustrates a syringe-to-syringe mixing system 100 for mixing a multi-phase fluoride varnish composition preparatory to applying it to a person's teeth. Mixing system 100 is one example of a closed vessel in which the multi-phase fluoride varnish composition may be mixed. Mixing system 100 includes a first syringe 102a coupleable to a second syringe 102b. First syringe 102a includes a first barrel 104a and a first plunger 106a slidably disposed within first barrel 104a. First barrel 104a and first plunger 106a are examples of a hollow cylindrical container and a plug, respectively. First plunger 106a forms a seal against the inner wall of first barrel 104a, allowing plunger 106a to push the contents of barrel 104a out of the barrel. Second syringe 102b includes a second barrel 104b and a second plunger 106b disposed within second barrel 104b. First syringe 102a and second syringe 102b are illustrated as being coupled together so as to allow the multi-phase fluoride varnish composition 108 to be cycled back and forth between the syringes so as to substantially suspend the solid fluoride salt phase within the liquid phase of the composition.

One example of means for coupling the syringes together may include threads formed near a distal end of first syringe 102a and corresponding receiving grooves formed near a distal end of second syringe 102b. Means for coupling may alternatively comprise any other suitable coupling mechanism formed at the distal ends of first and second syringes so as to allow the two syringes to be coupled together. Some specific coupling mechanisms are disclosed in U.S. Pat. No. 6,610,034, and U.S. patent application Ser. No. 11/235,461, filed Sep. 26, 2005 and entitled SYRINGE LOCKING STRUCTURES, both of which are hereby incorporated by reference with respect to their disclosure of structures for coupling two syringes together.

Because the multi-phase fluoride varnish composition 108 is advantageously contained within coupled syringes 102a and 102b, the composition 108 can be easily mixed so as to suspend the fluoride salt. Because the system 100 is closed, spillage of the composition 108 is advantageously prevented during mixing and suspension of the fluoride salt. Furthermore, the absence of any air within the closed system advantageously allows suspension of the solid phase without risk that a substantial quantity of air bubbles may become entrained within the composition.

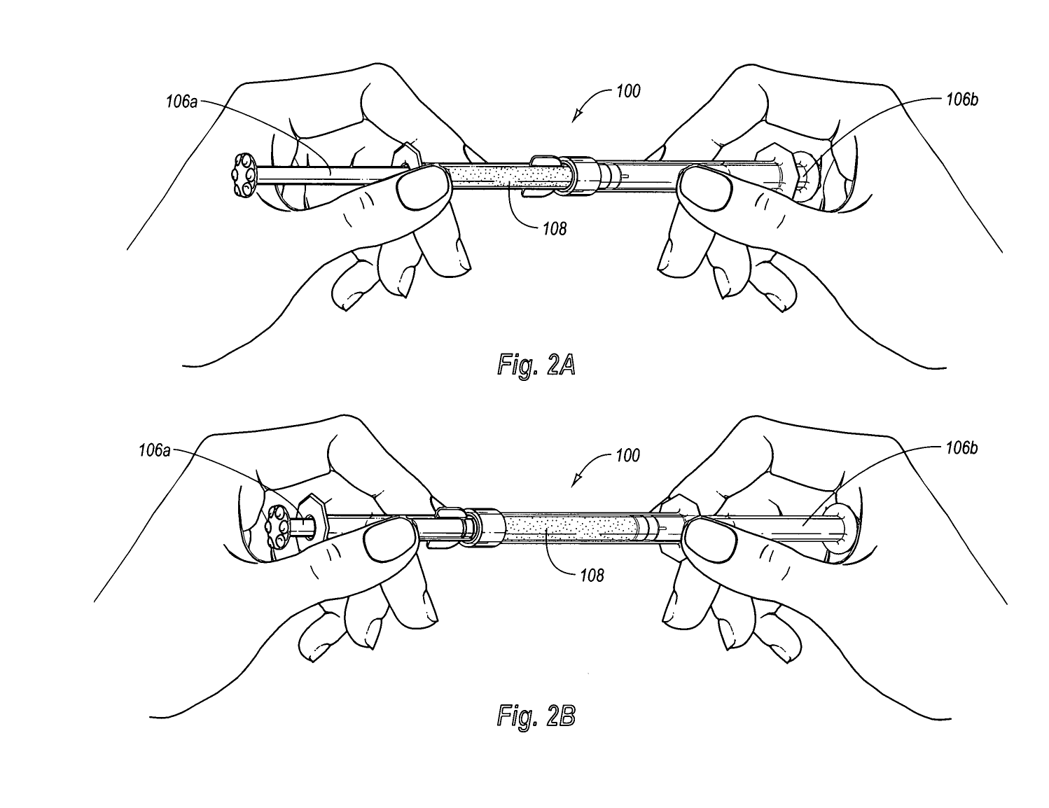

As illustrated in FIGS. 2A and 2B, the dental practitioner is advantageously able to apply turbulence to the multi-phase fluoride varnish composition 108 within first and second syringes of system 100 by manipulating plungers 106a and 106b so as to cycle the multi-phase fluoride varnish composition 108 back and forth. In such a mixing system the fluoride varnish composition may be cycled back and forth as many times as desired so as to substantially suspend the solid fluoride salt phase within the liquid phase of the composition. In one embodiment, the fluoride varnish composition may be cycled back and forth between about two and about fifteen times, preferably between about three and about twelve times, and more preferably between about five and about ten times.

Because the fluoride varnish composition 108 is advantageously contained within the coupled syringes, the composition 108 can be easily and efficiently mixed so as to suspend the fluoride salt without spilling composition 108, and without requiring the use of any additional tools (e.g., a stirring stick). Because the system 100 is closed, spillage of the composition 108 is advantageously prevented during mixing and suspension of the fluoride salt.

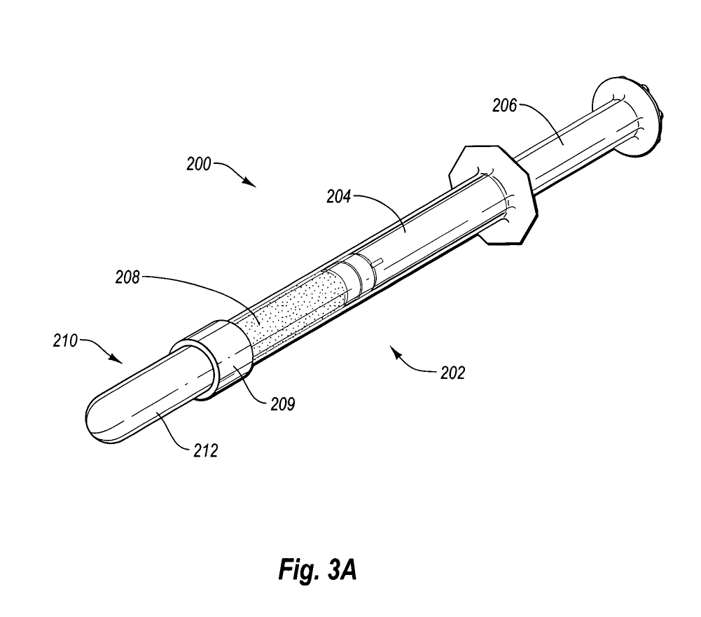

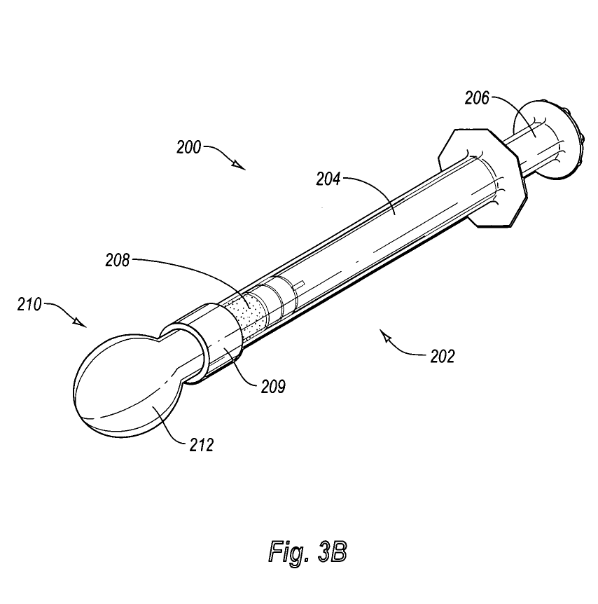

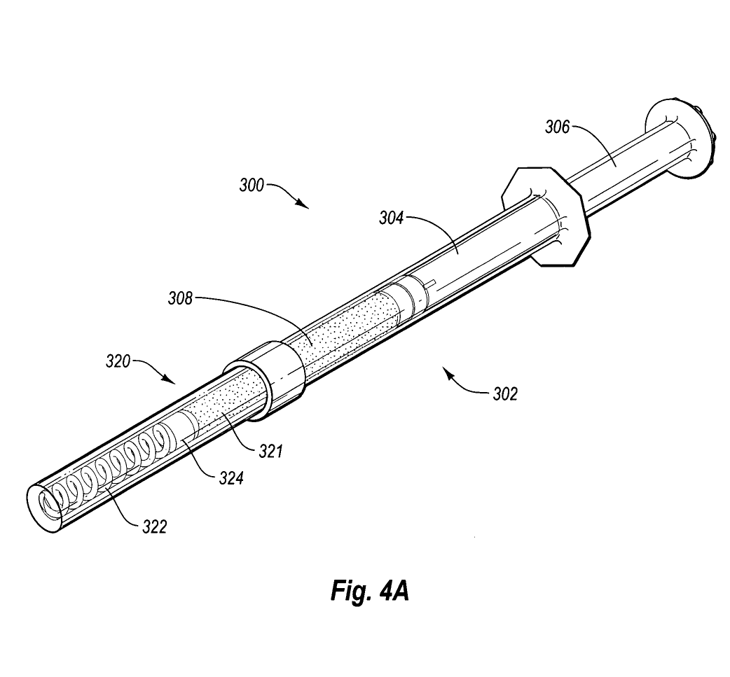

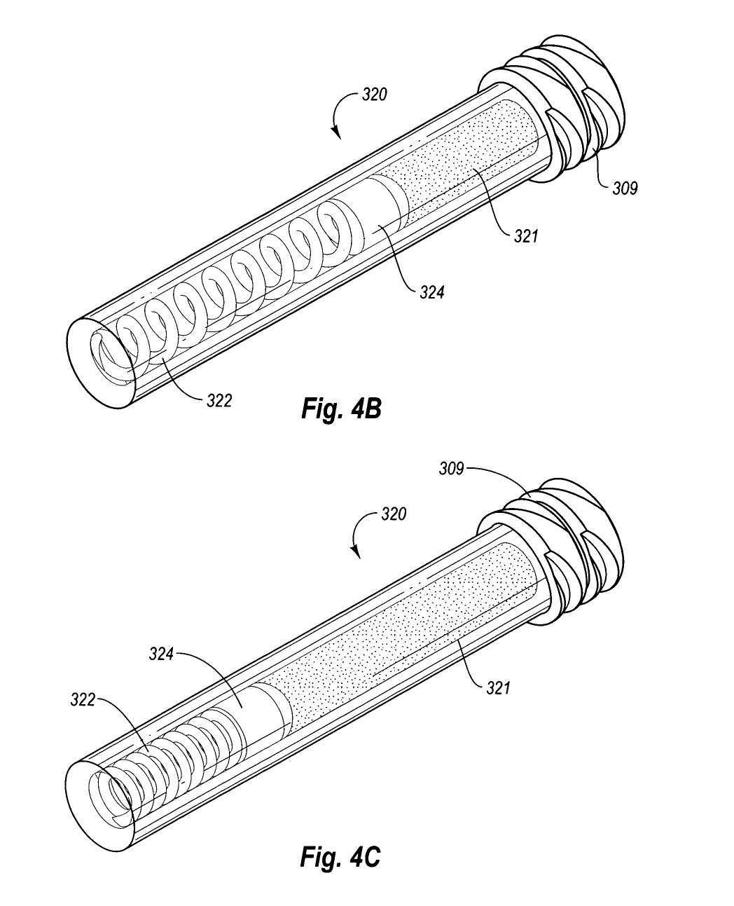

In addition, the closed vessel system 100 may advantageously be configured so as to contain a single use quantity of the multi-phase fluoride varnish composition 108. Such a system advantageously provides a single use system that can be provided to the dental practitioner as a uni-dose system which can be mixed and applied to the teeth of a single patient, after which the empty syringe-to-syringe mixing apparatus can be discarded. Any of the embodiments described below in conjunction with FIGS. 3A-3B and 4A-4C may also be configured so as to contain a single use quantity of fluoride varnish composition.

FIGS. 3A and 3B illustrate an alternative closed vessel mixing system 200 for mixing a multi-phase fluoride varnish composition preparatory to applying it to a person's teeth. Mixing system 200 is another example of a closed vessel in which the multi-phase fluoride varnish composition can be advantageously mixed while preventing spillage of the composition. Mixing system 200 includes a syringe 202 coupleable to a flexible bladder member 210. Syringe 202 includes a barrel 204 and a plunger 206 slidably disposed within barrel 204 so as to form a seal against the inner wall of barrel 204. Barrel 204 and plunger 206 are examples of a hollow cylindrical container and a plug, respectively. Flexible bladder member 210 advantageously includes coupling means 209 (e.g., male threads or corresponding engaging female grooves) near the proximal end of flexible bladder member 210 for coupling bladder member 210 to the distal end of syringe 202. Some exemplary coupling means were described above in conjunction with FIG. 1.

Bladder member 210 also includes a flexible balloon portion 212 (e.g., formed of an elastomeric material), which receives and fills with the varnish composition 208 when plunger 206 is pressed inward relative to barrel 204. In one embodiment, the balloon 212 may advantageously be sufficiently stiff so as to create a sufficient back pressure so as to push plunger 206 back outwardly relative to barrel 204 once composition 208 has filled balloon 212. In such an embodiment, this stiffness or bias causes the fluoride varnish composition 208 to remain at least partially within barrel 204 until plunger 206 is advanced inwardly relative to barrel 204 so as to push composition 208 into balloon portion 212 (see FIG. 3B). In other words, the balloon portion 212 may be biased so as to assume a deflated, unexpanded configuration as illustrated in FIG. 3A.

The dental practitioner is able to apply turbulence to the fluoride varnish composition 208 within system 200 by cycling the multi-phase fluoride varnish composition 208 back and forth between the barrel 204 and the balloon portion 212. Providing the balloon portion 212 with sufficient stiffness as described above advantageously allows this cycling to be accomplished by manipulating only the plunger 206, as the stiffness of the balloon material applies the back pressure necessary to force composition 208 back into barrel 204. The multi-phase fluoride varnish composition 208 may be cycled back and forth as many times as desired so as to substantially suspend the solid fluoride salt phase within the liquid adhesive phase of the composition. Because the fluoride varnish composition 208 is advantageously contained within closed system 200, the composition 208 can be easily mixed so as to suspend the fluoride salt, without risk that the composition may spill during mixing. Furthermore, the absence of any air within the closed system advantageously allows suspension of the solid phase without risk that a substantial quantity of air bubbles may become entrained within the composition.

FIGS. 4A-4C illustrate another alternative closed vessel mixing system 300 for mixing a multi-phase fluoride varnish composition 308 preparatory to applying it to a person's teeth. Mixing system 300 is another example of a closed vessel in which the fluoride varnish composition can be mixed. As seen in FIG. 4A, mixing system 300 includes a syringe 302 coupled to a spring loaded barrel 320. Syringe 302 includes a barrel 304 and a plunger 306 slidably disposed within barrel 304. Barrel 304 and plunger 306 are examples of a hollow cylindrical container and a plug, respectively. Plunger 306 forms a seal against the inside wall of barrel 304.

As perhaps best seen in FIGS. 4B-4C, spring loaded barrel 320 includes coupling means (e.g., threads or corresponding engaging grooves) 309 near proximal end of barrel 320. Spring loaded barrel 320 advantageously includes a coiled spring member 322 within barrel 321. The spring member 322 is biased against a plug 324. Spring 322 biases plug 324 to a substantially mid-way position within barrel 321 equal to the length of coiled spring member 322, as seen in FIG. 4B. FIG. 4B illustrates the coiled spring member 322 in an expanded configuration, while FIG. 4C illustrates spring member 322 under tension. When plunger 306 is pressed inward relative to barrel 304, the composition 308 is forced into barrel 321, moving plug 324 deeper within barrel 321 and compressing spring member 322, as seen in FIG. 4C. Plug 324 advantageously forms a seal against the inner wall of barrel 321, so as to prevent composition 308 from leaking into the portion of barrel 321 occupied by spring member 322. The spring may advantageously be configured to have sufficient tension to push composition 308 and plunger 306 back outwardly relative to barrel 304. Such a configuration is advantageous as it eliminates any need for the dental practitioner to pull the plunger 306 back outwardly relative to syringe barrel 304 in order to cycle the composition 308 back and forth between barrel 304 and barrel 321.

The dental practitioner is able to apply turbulence to the multi-phase fluoride varnish composition 308 within system 300 by alternatingly pressing and releasing plunger 306 so as to cycle the multi-phase fluoride varnish composition back and forth between barrel 304 and spring loaded barrel 320. In such a mixing system the fluoride varnish composition 308 may be cycled back and forth as many times as desired so as to substantially suspend the solid fluoride salt phase within the liquid adhesive phase. Because the spring loaded barrel 320 includes a spring member 322 as described above, cycling the composition 308 may advantageously be accomplished by manipulating only the plunger 306, as the spring member 322 applies the back pressure necessary to force composition 308 back into barrel 304. The fluoride varnish composition 308 may be cycled back and forth as many times as desired so as to substantially suspend the fluoride salt within the composition. Because the fluoride varnish composition 308 is advantageously contained within closed system 300, the composition 308 can be easily mixed so as to suspend the fluoride salt, without risk that the composition may spill during mixing.

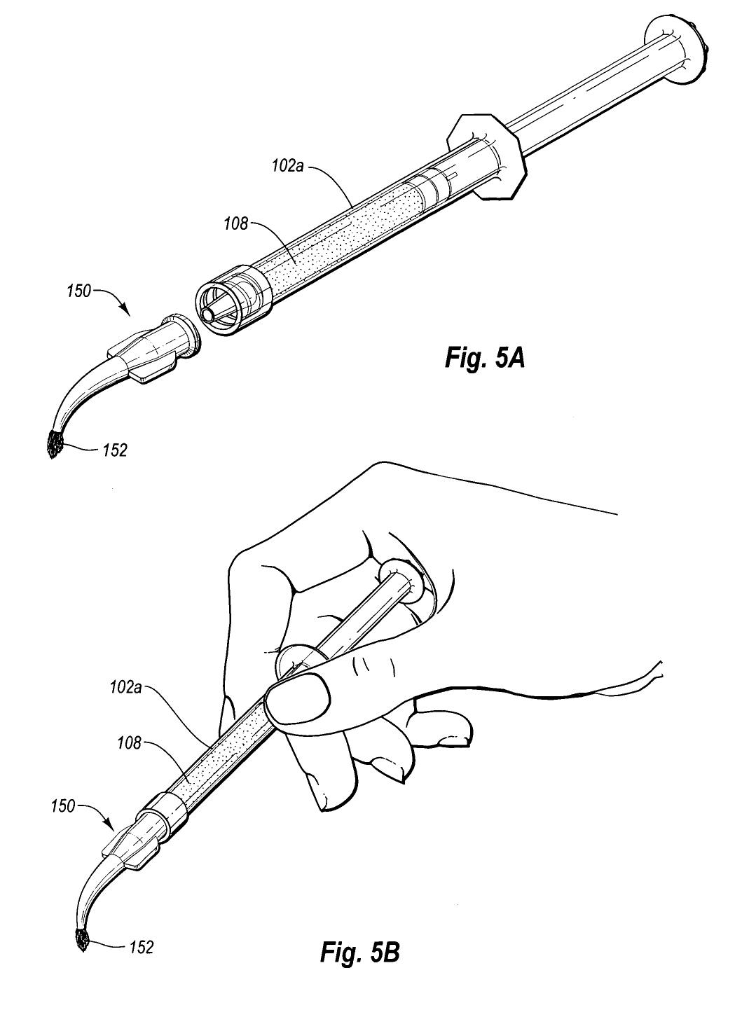

Once the fluoride salt has been substantially suspended (e.g., by any of the above described systems or another closed vessel system), the multi-phase fluoride varnish composition may then be applied to a person's teeth. According to one method, as seen in FIGS. 5A-5B, an applicator tip 150 may be coupled to syringe 102a containing the fluoride varnish composition 108 after the syringe has been separated from the remainder of the mixing system (e.g., the second syringe 102b of system 100, the flexible bladder member 210 of system 200, or the spring loaded barrel 320 of system 300). Tip 150 may be coupled to first syringe 102a so as to allow the dental practitioner to dispense fluoride varnish composition 108 onto a person's teeth. As illustrated, applicator tip 150 may include a flocked tip 152 so as to allow the user to paint the composition 108 onto the teeth. Use of an applicator tip 150 including a flocked tip 152 may be particularly preferred, although other types of tips (e.g., a tip including a foam pad, a cotton swab, or other absorbent material) may be used. A flocked tip 152 may be particularly preferred as the soft, flexible bristles of a flocked tip allow the composition to be painted onto a tooth surface in a substantially even (i.e., smooth) layer. Preferably, the composition including the suspended solid fluoride salt phase does not include a substantial quantity of air (e.g., greater than about 5% by volume) entrained within the composition at the time of application. The presence of a substantial quantity of air bubbles may make it difficult to apply the composition in a smooth and even layer because the air bubbles may cause the composition to assume an uneven configuration adjacent the bubbles, particularly those near the surface of the applied layer. In addition, bubbles that subsequently pop can cause the relatively viscous composition to assume an uneven configuration (e.g., the composition may be thicker or thinner relative to the adjacent composition thickness).

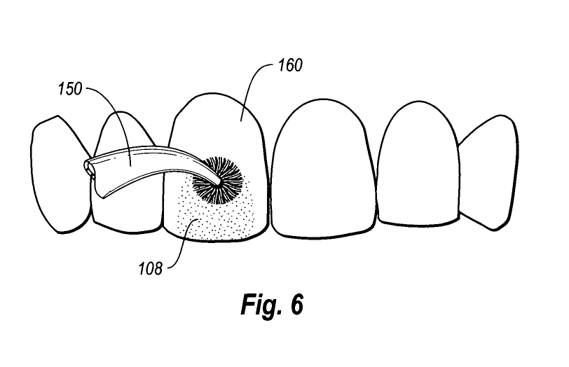

FIG. 6 illustrates the dental practitioner using the applicator tip 150 to apply the multi-phase fluoride varnish composition 108 to a person's tooth 160. As illustrated, the multi-phase fluoride varnish composition 108 may provide a visual contrast against the surface of the person's teeth so as to allow a dental practitioner to easily determine where the composition has been applied. Suspension of the solid fluoride salt phase provides an even distribution of the fluoride salt as applied to the teeth, providing a more consistent fluoride concentration and treatment. The fluoride varnish composition may be formulated so as to remain adhered to a person's teeth for any desired time. In one example, the fluoride varnish composition may remain adhered to the person's teeth so as to provide fluoride treatment to the teeth for at least about eight hours, preferably for at least about one day, and more preferably for at least about two days. The composition may remain adhered to the interproximal spaces between the teeth for even longer (e.g., as long as 7-10 days). Applying the composition so as to remain adhered for an extended period to the interproximal spaces between the teeth may be particularly advantageous as these locations are often where tooth decay is most likely to occur. Formulating and applying the composition so as to remain adhered for an extended time provides for an extended fluoride treatment time, which is a distinct advantage over compositions that may provide fluoride treatment for a much shorter time period.

### IV. EXAMPLES

### Example 1

A multi-phase fluoride varnish composition was formed by mixing together the following components:

| | |
|---|---|
| Sodium Fluoride | 5% |
| Poly Pale Resin (a partially dimerized rosin) | 44.25% |
| Refined Pharmaceutical Glaze(⅓ shellac and ⅔ ethanol) | 44.25% |
| Aerosil 200 (fumed silica) | 3% |
| Powdered Sucralose | 0.5% |
| Bubblegum flavor | 3% |

The sodium fluoride composition was yellow in color, and had a viscosity of about 2200 centipoise as measured using a Brookefield viscometer. The sodium fluoride varnish composition was introduced within a syringe-to-syringe closed mixing system. The sodium fluoride was substantially insoluble within the composition, settling to the bottom of the syringe barrels. A user cycled the material back and forth about 5 times in order to apply turbulence to the composition within the closed vessel so as to substantially suspend the fluoride salt within the composition. The composition was then applied to a patient's teeth. The composition remained adhered to the patient's teeth for about two days before wearing off. The composition applied to the interproximal spaces remained even longer.

### Example 2

A multi-phase fluoride varnish composition was formed by mixing together the following components:

| | |
|---|---|
| Sodium Fluoride | 5% |
| Canadian Balsam | 75% |
| Ethanol | 10% |
| Xylitol | 10% |

The sodium fluoride varnish composition was introduced within a syringe-to-syringe closed mixing system. The sodium fluoride was substantially insoluble within the composition, settling to the bottom of the syringe barrels. A user cycled the material back and forth about 5 times in order to apply turbulence to the composition within the closed vessel so as to substantially suspend the fluoride salt within the composition. The composition was then applied to a patient's teeth. The composition remained adhered to the patient's teeth for at least about two days before wearing off. The composition applied to the interproximal spaces remained even longer.

### Example 3

A multi-phase fluoride varnish composition is formed by mixing together the following components:

| | |
|---|---|
| Sodium Fluoride | 1% |
| Poly Pale Resin | 49% |
| Refined Pharmaceutical Glaze | 50% |

The sodium fluoride varnish composition is introduced within a syringe-to-syringe closed mixing system. The sodium fluoride is substantially insoluble within the composition, settling to the bottom of the syringe barrels. A user cycles the material back and forth about 5 times in order to apply turbulence to the composition within the closed vessel so as to substantially suspend the fluoride salt within the composition. The composition is then applied to a patient's teeth. The composition remains adhered to the patient's teeth for at least about two days before wearing off. The composition applied to the interproximal spaces remains even longer.

### Example 4

A multi-phase fluoride varnish composition is formed by mixing together the following components:

| | |
|---|---|
| Sodium Fluoride | 10% |
| Poly Pale Resin | 45% |
| Refined Pharmaceutical Glaze | 45% |

The sodium fluoride varnish composition is introduced within a syringe-to-syringe closed mixing system. The sodium fluoride is substantially insoluble within the composition, settling to the bottom of the syringe barrels. A user cycles the material back and forth about 5 times in order to apply turbulence to the composition within the closed vessel so as to substantially suspend the fluoride salt within the composition. The composition is then applied to a patient's teeth. The composition remains adhered to the patient's teeth for at least about two days before wearing off. The composition applied to the interproximal spaces remains even longer.

### Example 5

A multi-phase fluoride varnish composition is formed by mixing together the following components:

| | |
|---|---|
| Sodium Fluoride | 3% |
| Poly Pale Resin | 44.25% |
| Refined Pharmaceutical Glaze | 44.25% |
| Aerosil 200 | 3% |
| Xylitol | 2.5% |
| Grape flavor | 3% |

The sodium fluoride varnish composition is introduced within a syringe-to-syringe closed mixing system. The sodium fluoride is substantially insoluble within the composition, settling to the bottom of the syringe barrels. A user cycles the material back and forth about 5 times in order to apply turbulence to the composition within the closed vessel so as to substantially suspend the fluoride salt within the composition. The composition is then applied to a patient's teeth. The composition remains adhered to the patient's teeth for at least about two days before wearing off. The composition applied to the interproximal spaces remains even longer.

### Example 6

A multi-phase fluoride varnish composition is formed by mixing together the following components:

| | |
|---|---|
| Sodium Fluoride | 5% |
| Poly Pale Resin | 44.25% |
| Refined Pharmaceutical Glaze | 44.25% |
| Aerosil 200 | 3% |
| Powdered Sucralose | 0.5% |
| Bubblegum flavor | 3% |

The sodium fluoride varnish composition is introduced within a closed mixing system including a single syringe coupled to a flexible bladder as described in conjunction with FIGS. 3A-3B. The sodium fluoride is substantially insoluble within the composition, settling to the bottom of the syringe barrel. A user cycles the material back and forth about 5 times between the syringe barrel and the flexible bladder in order to apply turbulence to the composition within the closed vessel so as to substantially suspend the fluoride salt within the composition. The composition is then applied to a patient's teeth. The composition remains adhered to the patient's teeth for at least about two days before wearing off. The composition applied to the interproximal spaces remains even longer.

### Example 7

A multi-phase fluoride varnish composition is formed by mixing together the following components:

| | |
|---|---|
| Sodium Fluoride | 5% |
| Poly Pale Resin | 44.25% |
| Refined Pharmaceutical Glaze | 44.25% |
| Aerosil 200 | 3% |
| Powdered Sucralose | 0.5% |
| Bubblegum flavor | 3% |

The sodium fluoride varnish composition is introduced into a closed vessel mixing system including a spring loaded barrel coupled to a syringe as described in conjunction with FIGS. 4A-4C. The sodium fluoride is substantially insoluble within the composition, settling to the bottom of the syringe barrel. A user cycles the material back and forth about 5 times between the syringe barrel and the spring loaded barrel in order to apply turbulence to the composition within the closed vessel so as to substantially suspend the fluoride salt within the composition. The composition is then applied to a patient's teeth. The composition remains adhered to the patient's teeth for at least about two days before wearing off. The composition applied to the interproximal spaces remains even longer.

It will be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A method for mixing a multi-phase fluoride varnish composition preparatory to applying it to a person's teeth, comprising:
providing a multi-phase fluoride varnish composition comprising a liquid hydrophobic adhesive phase and a solid fluoride salt phase that is substantially insoluble in the liquid hydrophobic adhesive phase, the multi-phase fluoride varnish composition being contained within a closed vessel; and
applying turbulence to the multi-phase fluoride varnish composition within the closed vessel in order to substantially suspend the solid fluoride salt phase within the liquid hydrophobic adhesive phase while preventing spillage of the varnish composition.

2. A method as recited in claim 1, wherein the closed vessel comprises a hollow cylindrical container including an inner wall and a plug slidably disposed within the hollow cylindrical container, the plug forming a seal against the inner wall, and wherein the step of applying turbulence to the multi-phase fluoride varnish composition comprises cycling the composition in and out of the hollow cylindrical container with the plug.

3. A method as recited in claim 2, wherein the multi-phase fluoride varnish composition is cycled in and out between about 2 and about 15 times.

4. A method as recited in claim 2, wherein the multi-phase fluoride varnish composition is cycled in and out between about 3 and about 12 times.

5. A method as recited in claim 2, wherein the multi-phase fluoride varnish composition is cycled in and out between about 5 and about 10 times.

6. A method as recited in claim 2, wherein the hollow cylindrical container comprises a syringe barrel of a first syringe and the plug comprises a plunger of a first syringe, the plunger being slidably disposed within the syringe barrel such that the closed vessel comprises a first syringe.

7. A method as recited in claim 6, wherein the closed vessel further comprises a second syringe coupleable to the first syringe, the closed vessel comprising a syringe-to-syringe mixing apparatus when the first syringe is coupled to the second syringe, the step of applying turbulence comprising cycling the multi-phase fluoride varnish composition back and forth between the first syringe and the second syringe of the syringe-to-syringe mixing apparatus.

8. A method as recited in claim 6, wherein the closed vessel further comprises a flexible bladder coupleable to a distal end of the first syringe, the step of applying turbulence comprising cycling the multi-phase fluoride varnish composition back and forth between the first syringe and the flexible bladder in order to substantially suspend the solid fluoride salt phase within the liquid hydrophobic adhesive phase.

9. A method as recited in claim 6, wherein the closed vessel further comprises a spring loaded barrel coupleable to a distal end of the first syringe, the step of applying turbulence comprising cycling the multi-phase fluoride varnish composition back and forth between the syringe barrel and the spring loaded barrel in order to substantially suspend the solid fluoride salt phase within the liquid hydrophobic adhesive-phase.

10. A method as recited in claim 9, wherein the multi-phase fluoride varnish composition is cycled back and forth between the syringe and the spring loaded barrel by alternatingly pressing and releasing the plunger of the first syringe.

11. A method as recited in claim 1, further comprising applying the multi-phase fluoride varnish composition to a person's teeth.

12. A method as recited in claim 11, wherein the multi-phase fluoride varnish composition provides a visual contrast against the surface of the person's teeth so as to allow a dental practitioner to easily determine where the multi-phase fluoride varnish composition has been applied.

13. A method as recited in claim 11, wherein the multi-phase fluoride varnish composition remains adhered to the person's teeth so as to provide fluoride treatment to the teeth for at least about 8 hours.

14. A method as recited in claim 11, wherein the multi-phase fluoride varnish composition remains adhered to the person's teeth so as to provide fluoride treatment to the teeth for at least about one day.

15. A method as recited in claim 11, wherein the multi-phase fluoride varnish composition remains adhered to the person's teeth so as to provide fluoride treatment to the teeth for at least about two days.

16. A method as recited in claim 1, wherein the multi-phase fluoride varnish composition has a viscosity of at least about 1000 centipoise.

17. A method as recited in claim 1, wherein the multi-phase fluoride varnish composition has a viscosity between about 1300 centipoise and about 3500 centipoise.

18. A method as recited in claim 1, wherein the multi-phase fluoride varnish composition has a viscosity between about 1700 centipoise and about 2800 centipoise.

19. A system for mixing a multi-phase fluoride varnish composition preparatory to applying it to a person's teeth, comprising:
a closed vessel for holding a multi-phase fluoride varnish composition;
a quantity of a multi-phase fluoride varnish composition comprising a liquid hydrophobic adhesive phase and a solid fluoride salt phase that is insoluble in the liquid hydrophobic adhesive phase, the quantity of multi-phase fluoride varnish composition being contained within the closed vessel; and
means for providing turbulence to the multi-phase fluoride varnish composition within the closed vessel so as to substantially suspend the solid fluoride salt phase within the liquid hydrophobic adhesive phase while preventing spillage of the varnish composition.

20. A system as recited in claim 19, wherein the closed vessel comprises a hollow cylindrical container including an inner wall and the means for providing turbulence comprises a plug slidably disposed within the hollow cylindrical container, the plug forming a seal against the inner wall.

21. A system as recited in claim 20, wherein the hollow cylindrical container comprises a syringe barrel of a first syringe and the plug comprises a plunger of a first syringe, the plunger being slidably disposed within the syringe barrel such that the closed vessel comprises a first syringe.

22. A system as recited in claim 21, wherein the closed vessel further comprises a second syringe coupleable to the first syringe, the closed vessel comprising a syringe-to-syringe mixing apparatus when the first syringe is coupled to the second syringe.

23. A system as recited in claim 21, wherein the closed vessel further comprises a flexible bladder coupleable to a distal end of the first syringe.

24. A system as recited in claim 23, wherein the flexible bladder is sufficiently stiff so as to be biased to a deflated configuration.

25. A system as recited in claim 21, wherein the closed vessel further comprises a spring loaded barrel coupleable to a distal end of the first syringe.

26. A system as recited in claim 19, wherein the closed vessel contains a single use quantity of the multi-phase fluoride varnish composition such that the closed vessel may be discarded after a single use.

27. A system as recited in claim 19, wherein the liquid hydrophobic adhesive phase comprises at least one of shellac or colophonium.

28. A system as recited in claim 19, wherein the multi-phase fluoride varnish composition further comprises at least one thickener.

29. A system as recited in claim 28, wherein the thickener comprises fumed silica.

30. A system as recited in claim 19, wherein the multi-phase fluoride varnish composition further comprises at least one sweetener.

31. A system as recited in claim 19, wherein the multi-phase fluoride varnish composition further comprises at least one flavorant.

32. A system as recited in claim 19, wherein the solid fluoride salt phase comprises sodium fluoride.

33. A system as recited in claim 19, wherein the solid fluoride salt phase is included in an amount between about 0.1% and about 10% by weight of the multi-phase fluoride varnish composition.

34. A system as recited in claim 19, wherein the solid fluoride salt phase is included in an amount between about 1% and about 8% by weight of the multi-phase fluoride varnish composition.

35. A system as recited in claim 19, wherein the solid fluoride salt phase is included in an amount between about 3% and about 7% by weight of the multi-phase fluoride varnish composition.

36. A system as recited in claim 19, wherein the multi-phase fluoride varnish composition has a viscosity of at least about 1000 centipoise.

37. A system as recited in claim 19, wherein the multi-phase fluoride varnish composition has a viscosity between about 1300 centipoise and about 3500 centipoise.

38. A system as recited in claim 19, wherein the multi-phase fluoride varnish composition has a viscosity between about 1700 centipoise and about 2800 centipoise.

39. A system for mixing a multi-phase fluoride varnish composition preparatory to applying it to a person's teeth, comprising:
a closed vessel comprising a hollow cylindrical container including an inner wall;
a quantity of a multi-phase fluoride varnish composition comprising a liquid hydrophobic adhesive phase and a solid fluoride salt phase that is insoluble in the liquid hydrophobic adhesive phase, the quantity of multi-phase fluoride varnish composition being contained within the closed vessel; and
a plug disposed within the hollow cylindrical container, the plug forming a seal against the inner wall, the plug being slidably disposed within the hollow cylindrical container so as to selectively provide turbulence to the multi-phase fluoride varnish composition within the closed vessel so as to substantially suspend the solid fluoride salt phase within the liquid hydrophobic adhesive phase.
